(19) United States
(12) Patent Application Publication
(10) Pub. No.: US 2009/0328249 A1
(43) Pub. Date: Dec. 31, 2009

(54) Transplastomic Plants Expressing Lumen-Targeted Protein

(76) Inventors: Eric Paget, Caluire (FR); Manuel Dubald, Saint Didier Au Mont D'or (FR); Julia Bally, Lyon (FR)

Correspondence Address: CONNOLLY BOVE LODGE & HUTZ, LLP, P O BOX 2207, WILMINGTON, DE, 19899, US

(21) Appl. No.: 12/307,815

(22) PCT Filed: Jul. 2, 2007
(86) PCT No.: PCT/EP07/56620

§ 371 Date: Jan. 29, 2009

(30) Foreign Application Priority Data
Jul. 7, 2006 (EP) 06356090.8

(51) Int. Cl. A01H 5/00; A01H 13/00; C12N 15/82; C07H 21/04; C12N 15/63; C12N 5/10; C12P 21/06

(52) U.S. Cl. 800/278; 800/298; 800/296; 800/317.3; 800/317.2; 800/317.4; 800/306; 800/314; 800/312; 536/23.1; 435/320.1; 435/419; 435/69.1

(57) ABSTRACT

The present invention relates to nucleic acid sequences and methods useful in targeting a recombinant protein encoded by a transgene integrated into the chloroplast genome to the thylakoid lumen of chloroplast, whereby said nucleic acid sequences encode bacterial signal peptides. The invention also relates to means and methods for expressing a disulfide-bond containing protein of interest in a transplastomic plant cell.

The present invention relates to new constructs and methods for the expression of recombinant proteins in the thylakoid lumen of transplastomic plant cells. The invention also relates to means and methods for expressing a disulfide-bond containing protein of interest in a transplastomic plant cell.

Plants offer a suitable alternative to microbial or animal expression for the production of recombinant industrial or pharmaceutical proteins. They present many advantages compared with traditional systems such as anticipated lower production costs, rapid scalability, absence of human pathogens and ability to fold and assemble complex proteins accurately (Ma et al., 2003, Nature reviews. 4:794-802). A number of model plants have been successfully transformed to produce complex structures with native conformation such as antibodies or complex antigens (Warzecha and Mason, 2003, J Plant Physiol. 160(7): 755-64; Arntzen et al., 2005, vaccine. 23:1753-1756). Moreover, some plant species have the potential to be suitable directly for oral immunization, the edible vaccine concept, including of humans (Mason et al., 2002, Trends in Mol. Med. 8(7):324-329). However there are some limitations since nuclear transformation of plants often results in low yield of the recombinant protein.

As an alternative to nuclear expression, chloroplast genetic engineering has emerged as an effective tool for the expression of recombinant proteins in plants (Daniell et al., 2004, In molecular Biology of Plant organelles, Springer, 443-490; Maliga 2004, Annu. Rev. Plant. Biol. 55:289-313; Dubald et al., 2006, In recent Advances in Plant Biotechnology, Kumar, A. (Ed), IK International Publishers. New Dehli (in press)). The main reason is that this system is characterized by its potential for very high level expression of the transgene, up to 46% total soluble proteins (De Cosa et al., 2001, Nat. biotechnol. 19:71-74). Additional features of interest are (i) the maternal inheritance of its genome in most species, reducing drastically transgene dissemination via the pollen, and (ii) the targeted integration of introduced DNA by homologous recombination into a defined region of the plastid genome. The location of the transgenes is thus predictable, gene expression is uniform among the selected transgenic lines which are clonal in essence, and there is no segregation of the character in the progeny.

Plastids are eukaryotic cell organelles which according to the endosymbiotic theory derive from cyanobacterial ancestors. They therefore still exhibit many prokaryotic features, including for example gene organization in operons, and most prokaryotic mechanisms of gene expression.

At the protein level, it was anticipated that chloroplasts, like other prokaryotic systems, would not be able to accumulate recombinant proteins containing correctly formed disulfide bonds, which represent an important class of therapeutic proteins. There are so far in the literature only a few examples of disulfide-bond containing recombinant proteins which have been successfully expressed in plastids (Staub, J. M., et al, 2000; Daniell et al., 2001). Nevertheless, it is not always clear whether these bonds are correctly paired, and if they are formed in planta or spontaneously during extraction.

Chloroplasts are complex organelles in structural terms, comprising three distinct soluble phases. The chloroplast is bound by a double-membrane envelope, which encloses an intermembrane space. The major soluble phase is the stroma, which is the site of carbon fixation, amino acids synthesis and many other pathways. The dominant membrane is the extensive interconnecting thylakoid network, where light is captured and ATP synthesized. The thylakoid membrane encloses the third soluble phase, the thylakoid lumen, which houses a number of extrinsic photosynthetic proteins as well as many others (C. Robinson et al, 2001, Traffic 2:245-251).

The thylakoid lumen of chloroplasts is a plant cellular compartment which might be optimal for the accumulation of certain recombinant proteins due to its particularity, including its particular content in proteases (Z. Adams et al., TRENDS in Plant science, vol 7 No 10, pp 451-456, 2002). Despite this, it has rarely been considered for recombinant protein targeting and accumulation.

In the U.S. Pat. No. 6,512,162, the aprotinin coding sequence is fused with the petA gene in order to target the fused petA::aprotinin protein to the plant cell thylakoid membrane. PetA is a gene from the chloroplast genome encoding the cytochrome f (petA) protein, which has been reported as a polypeptide with a transmembrane arrangement in the chloroplast thylakoid membrane, with the N-terminal region in the intrathylakoid space, and a 15 amino acid C terminal sequence in the stroma (S. J. Rothstein et al, Proc. Natl. Acad. Sci. USA, Vol 82 pp 7955-7959, 1985). Therefore, the fused petA::aprotinin protein wherein the aprotinin coding sequence is linked to the 3′ terminus of the coding sequence of cytochrome f (petA) should address the aprotinin in the stroma.

There is therefore still a need for methods and means which address a peptide of interest in the thylakoid lumen of chloroplast.

Such a strategy takes advantage of the high-level expression for transgenes integrated into the chloroplast genome in order to accumulate high amount of recombinant protein in a cell compartment (the thylakoid lumen) with particular characteristics.

### DESCRIPTION OF THE FIGURES

FIG. 1: map of plasmid pCLT 516

### DESCRIPTION OF THE INVENTION

The present invention provides nucleic acid sequences useful in targeting a recombinant protein encoded by a transgene integrated into the chloroplast genome to the thylakoid lumen of chloroplast, whereby said nucleic acid sequences encode bacterial signal peptides.

Furthermore, a remarkable and unexpected feature of using the method and means of the invention is the great enhancement in the expression and in the specific activity of the recombinant protein.

The present invention further relates to means and methods for obtaining a disulfide-bond-containing protein of interest in the thylakoid lumen of a transplastomic plant cell, and/or for producing recombinant proteins having a non-methionine N-terminus in plant chloroplasts.

The subject of the invention is a chimeric gene comprising, linked to one another in a functional fashion in the direction of transcription:

a) a promoter from a plastomic plant gene,
b) a nucleic acid sequence encoding a bacterial signal peptide translationally fused with,
c) a heterologous nucleic acid sequence encoding a peptide of interest,
d) optionally a terminator which is active in the plastids of plant cells.

According to the invention, the “bacterial signal peptide” can be any signal peptide of bacterial origin, i.e. any signal peptide of secreted bacterial protein. A secreted bacterial protein is a protein of bacterial origin which is secreted by the bacteria outside the bacterial cell. Secreted bacterial proteins naturally contain a signal peptide that enables the bacterial cell machinery to target said protein outside the bacterial cell. The bacterial signal peptide of the invention can be an N-terminal signal peptide, i.e. a signal peptide of a bacterial protein located at the N-terminal end of said bacterial protein.

In bacteria, protein secretion depends mostly on N-terminal signal peptides. There are at least two distinct secretory pathways that both depend on N-terminal signal peptides: the Sec pathway and the Tat pathway. In bacteria, there is also a third kind of secretory mechanism called type III secretion or the “general secretory pathway”. Type III secretion signals are located at the C-terminal end of proteins.

The Sec pathway is, in bacteria, the major route of protein translocation. Secretory proteins are synthesized in the cytosol as precursors with an amino-terminal extension, the signal peptide. These precursor proteins (preproteins) are either targeted directly or via molecular chaperones to a membrane-bound complex termed “translocase”. Preproteins are translocated across the membrane in an unfolded state. Finally, at the periplasmic face of the membrane, the signal sequence of the preprotein is removed by a signal peptidase (A. J. M. Driessen, 2002, Protein Targeting, transport & translocation, R. E. Dalbey R. E. & G. von Heijne ed., chap 4-“protein export in bacteria”, 47-73).

The Tat pathway has been later found to exist in most bacteria. The hydrophobic region of Tat signal peptides have a lower average hydrophobicity than the ones in Sec signal peptides, and the Tat signal peptides are not recognized by the components of the Sec machinery. Nevertheless, and despite the fact that there is no apparent relationship between the components of the Sec and Tat machineries, the respective signal peptides are relatively similar in their overall design, and seem more like variations on a theme than two distinct kinds of targeting signals (G. von Heijne, 2002, Protein Targeting, transport & translocation, R. E. Dalbey R. E. & G. von Heijne ed., chap 3—“Targeting sequences”, 35-46)

Thereby, the present invention relates to a chimeric gene as defined above, wherein the nucleic acid sequence encoding a bacterial signal peptide is from a bacterial protein using the Sec pathway, or the Tat pathway, for its translocation.

Bacterial signal peptides are known to have a length that generally ranges from 18 up to 30 amino acids, but bacterial signal peptides of any length are suitable for the present invention. They are equipped with the same physical properties and generally have the following tripartite structure:
N-domain: the amino-terminal domain contains a net positive charge. Preproteins that do not have this positive charge are still recognized by the translocase but are translocated slowly. The known N-domains from 1 to 5 amino acids, but bacterial signal peptides with longer N-domains could also be suitable to the present invention;
H-domain: the hydrophobic core of the signal sequence consists of a stretch of hydrophobic amino acid residues that may fold into an alpha-helical conformation. Frequently, glycine and proline residues are found in the middle of this domain. The known N-domains contain from 7 to 15 amino acids, but bacterial signal peptides with H-domain of different length could also be suitable to the present invention;
C-domain: the polar C-domain contains the cleavage site for signal peptidases. The known N-domains contain from 3 to 7 amino acids, but bacterial signal peptides with C-domain of different length could also be suitable to the present invention; Prediction and identification of the signal peptide from a bacterial protein based on the amino acid sequence of the protein or on the nucleic acid sequence of the corresponding gene is well known to the skilled person.

As a non-limiting example, SignaIP (Nielsen et al., Int. J. Neural Syst. 8:581-599, 1997) and TargetP (Emanuelsson et al, J Mol Biol 300:1005-1016, 2000) are suitable tools to predict the signal peptide of a bacterial protein or the nucleic acid sequence encoding a bacterial signal peptide.

It is well known to the skilled person that normal translation in plastids initiates at methionine. In accordance with the invention, an ATG translation start codon, coding for a methionine, is fused in frame at the 5′ end of the nucleic acid molecule encoding a bacterial signal peptide or substituted to the N-terminus amino acid when such bacterial signal peptide does not start by a methionine and/or when said nucleic acid molecule encoding a lumen targeting signal peptide does not start by an ATG codon.

Nucleic acid molecules encoding a bacterial signal peptide may be isolated e.g. from DNA libraries produced from bacterial origin. Alternatively, they may have been produced by means of recombinant DNA techniques (e.g. PCR) or by means of chemical synthesis. The identification and isolation of such nucleic acid molecules may take place by using the molecules according to the invention or parts of these molecules or, as the case may be, the reverse complement strands of these molecules, e.g. by hybridization according to standard methods (see e.g. Sambrook et al., 1989, Molecular Cloning, A Laboratory Manual, 2nd Edition, Cold Spring Harbor Laboratory Press, Cold Spring Harbor, N.Y.).

A bacterial signal peptide according to the invention can be obtained from a bacterial protein using techniques which are familiar to those skilled in the art, notably methods such as those described in Sambrook et al (1989, Molecular Cloning, a Laboratory Manual, Nolan C., ed., New Yyork: Cold Spring Harbor Laboratory Press).

In an embodiment of the present invention the nucleic acid sequence encoding a bacterial signal peptide is chosen from the group consisting of:

a) Nucleic acid molecule which encodes the amino acid sequence comprising the amino acid sequence given under SEQ ID NO: 2;
b) Nucleic acid molecule which encodes a peptide, the amino acid sequence of which has an identity of at least 70%, at least 80%, at least 90% or 95% with the amino acid sequence given under SEQ ID NO:2;
c) Nucleic acid molecule, comprising the nucleotide sequence given under SEQ ID NO 1;
d) Nucleic acid molecule, the nucleic acid sequence of which has an identity of at least 50%, at least 60%, at least 70%, 80%, 90%, or 95% with the nucleic acid sequences described under a) or c);
e) Nucleic acid molecules, the nucleotide sequence of which deviates from the sequence of the nucleic acid molecules identified under a), b), c) or d) due to the degeneration of the genetic code; and
f) Nucleic acid molecules, which represent fragments, allelic variants and/or derivatives of the nucleic acid molecules identified under a), b), c), d) or e).

In accordance with the present invention, the term “identity” is to be understood to mean the number of amino acids/nucleotides corresponding with the amino acids/nucleotides of other protein/nucleic acid, expressed as a percentage. Identity is preferably determined by comparing the Seq. ID NO: 1, or SEQ ID NO: 2 with other protein/nucleic acid with the help of computer programs. If sequences that are compared with one another have different lengths, the identity is to be determined in such a way that the number of amino acids, which have the shorter sequence in common with the longer sequence, determines the percentage quotient of the identity. Preferably, identity is determined by means of the computer program ClustalW, which is well known and available to the public (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from different Internet sites, including the IGBMC (Institut de Génétique et de Biologie Moléculaire et Cellulaire, B. P. 163, 67404 Illkirch Cedex, France; ftp://ftp-igbmc.u-strasbg.fr/pub/) and the EBI (ftp://ftp.ebi.ac.uk/pub/software/) as well as from all mirrored Internet sites of the EBI (European Bioinformatics Institute, Wellcome Trust Genome Campus, Hinxton, Cambridge CB10 1SD, UK).

Preferably, Version 1.8 of the ClustalW computer program is used to determine the identity between proteins according to the invention and other proteins. In doing so, the following parameters must be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, Version 1.8 of the ClustalW computer program is used to determine the identity between the nucleotide sequence of the nucleic acid molecules according to the invention, for example, and the nucleotide sequence of other nucleic acid molecules. In doing so, the following parameters must be set: KTUPLE=2, TOPDIAGS=4, PAIRGAP=5, DNAMATRIX:IUB, GAPOPEN=10, GAPEXT=5, MAXDIV=40, TRANSITIONS: unweighted.

The closest relatives to the predecessors of chloroplasts known are cyanobacteria, that are capable of performing oxygenic photosynthesis. Like chloroplasts, cyanobacteria house an internal thylakoid membrane system which harbors the protein complexes of the photosynthetic electron transport chain. According to the invention, the bacterial signal peptide may be from a cyanobacteria origin or not, and/or from a protein located inside a bacterial thylakoid lumen or not.

In accordance with the present invention, the terms “linked to one another in a functional fashion” or “operably linked to” means that the specified elements of the component chimeric gene are linked to one another in such a way that they function as an unit to allow expression of the coding sequence. By way of example, a promoter is said to be linked to a coding sequence in a functional fashion if it is capable of promoting the expression of said coding sequence.

In accordance with the present invention, the terms ““linked to one another in a functional fashion” cover the case of a polycistronic arrangement wherein the promoter is not directly linked to the coding sequence.

A chimeric gene according to the invention can be assembled from the various components using techniques which are familiar to those skilled in the art, notably methods such as those described in Sambrook et al (1989, Molecular Cloning, a Laboratory Manual, Nolan C., ed., New york: Cold Spring Harbor Laboratory Press). Exactly which regulatory elements are to be included in the chimeric gene would depend on the plant and the type of plastid in which they are to work: those skilled in the art are able to select which regulatory elements are going to work and can improve the production of protein into a given plant. As an example, the Shine-Dalgarno (SD) consensus sequence GGAGG can be placed upstream of the gene. Alternatively or in addition, a 5′ untranslated region (UTR) can be inserted between the promoter and the gene (Staub J. M. and Maliga P., 1993, EMBO J. 12, 601-606).

Those skilled in the art are aware than the use of 5′ untranslated region (5′UTR) and 3′ untranslated region (3′UTR) regulatory signals are generally necessary for higher levels of transgene expression in plastids (De Cosa B., Moar W., Lee S. B., Miller M. and Daniell H., 2001, Nat. Biotechnol. 19, 71-74). Possible 5′UTR and 3′UTR are well known by those skilled in the art. As an example, the promoter of the psbA gene, nucleotide 1596 to 1819 from Genbank Z00044, includes the endogenous 5′UTR. The promoter of the 16S ribosomal operon Prrn can be associated with the ribosome binding site region of the rbcL gene (5′UTR rbcL).

In the context of the invention, a promoter from a plastomic plant gene means a promoter which is naturally present in the plastome of a plant.

Among the promoters from a plastomic plant gene, by way of example, special mention can be made of the psbA gene which encodes the D1 polypeptide of PSII (Staub et al. 1993 EMBO Journal 12(2):601-606), and the constitutive Prrn promoter which regulates the ribosomal RNA operon (Staub et al. 1992 Plant Cell 4:39-45). As a general rule, those skilled in the art will know which of the available promoters to select in order to obtain the desired mode of expression (constitutive or inducible).

A well-suited promoter for the current invention is the Prrn promoter of tobacco which is associated with part of the 5′ untranslated sequence of the rbcL gene providing a ribosome-binding site (Svab et al., 1993, Proc. Natl. Acad. Sci. 90:913-917).

Another well-suited promoter is the light-dependent promoter of the psbA gene which encodes the D1 polypeptide of PSII (Staub J. M. and Maliga P., 1993, EMBO J. 12, 601-606).

Among the terminators which are active in plant cell plastids, by way of example, special mention could be made of the terminators of the psbA gene, the rbcL gene (which codes for the large sub-unit of RuBisCO), and the rps16 gene (which codes for a tobacco ribosomal protein) (Shinozaki et al., 1986, EMBO J. 5:2043-2049; Staub J. M. and Maliga P., 1993, EMBO J. 12, 601-606).

In accordance with the present invention, the term “translationally fused with” shall mean a fusion of nucleic acid sequences in such a way that they represent a single open reading frame, which upon transcription leads to the production of a single messenger RNA encoding a single polypeptide, when translated.

In accordance with the present invention, the nucleic acid sequence encoding a bacterial signal peptide is translationally fused with a heterologous nucleic acid sequence, which means that this second nucleic acid sequence is not naturally fused with the first nucleic acid sequence encoding a bacterial signal peptide.

In another embodiment of the invention, the nucleic acid sequence encoding a bacterial signal peptide and/or the nucleic acid sequence encoding a peptide of interest are designed in order to optimize chloroplast expression, based for example on the chloroplast codon usage of Nicotiana tabacum. The chloroplast codon usage of Nicotiana tabacum is available on www. Kazusa.or.jp/codon, and the distribution of the codons is randomly attributed to each amino acid residue over the entire coding sequence according to the frequency in the chloroplast codon usage table (Nakamura et al., 2000, Nucl. Acids Res. 28, 292).

The nucleic acid sequence encoding a peptide of interest may be isolated e.g. from genomic DNA or DNA libraries produced from eukaryotic or other origin. Alternatively, they may have been produced by means of recombinant DNA techniques (e.g. PCR) or by means of chemical synthesis. The identification and isolation of such nucleic acid molecules may take place by using the molecules according to the invention or parts of these molecules or, as the case may be, the reverse complement strands of these molecules, e.g. by hybridization according to standard methods (see e.g. Sambrook et al., 1989, Molecular Cloning, A Laboratory Manual, 2nd Edition, Cold Spring Harbor Laboratory Press, Cold Spring Harbor, N.Y.).

In yet another embodiment of the invention, the nucleic acid sequence encoding a peptide of interest encodes a peptide having a non-methionine N-terminus.

Among the peptides of interest having a non-methionine N-terminus, by way of example, mention can be made of the aprotinin protein. Aprotinin is a protease inhibitor which can be extracted from bovine organs or tissues, such as pancreas, lungs, or liver. Aprotinin is known to inhibit various serine proteases, including trypsin, chymotrypsin, plasmin and kallikrein, and is used therapeutically in the treatment of the myocardial infarction, shock syndrome, hyperfibrinolytic and acute pancreatitis, and in order to reduce blood loss in connection with cardiac surgery (Bidstrup et al, 1989, Cardiovasc Surg. 44:640-645). The nucleic acid and amino acid sequences of aprotinin can be found in the Swiss-Prot/TrEMBL database (collaboration between the Swiss Institute of Bioinformatics and the EMBL outstation—the European Bioinformatics Institute; http://us.expasy.org/sprot) under the Accession Number P00974.

The invention also relates to a vector designed for the transformation of plant plastids, characterized in that it contains at least two sequences that are homologous to sequences in the plastome of the plant to be transformed, said homologous sequences flanking at least one chimeric gene according to the invention.

These sequences—one upstream (LHRR) and the other downstream (RHRR) of the component chimeric gene(s)—permit double homologous recombination within an intergenic region of the plastome, comprising the contiguous region LHRR and RHRR.

The two homologous recombination sequences according to the invention may be contiguous so that the chimeric gene is inserted at a non-coding (intergenic) sequence of the plastome. In a particular embodiment, this sequence is part of the operon of the plastid ribosomal RNA. In another particular embodiment, the non-coding sequence includes the 3′ end of the rbcl gene (which codes for the large subunit of ruBisCO), with the other homologous sequence including the 5′ end of the accD gene (which codes for one of the subunits of acetyl-CoA carboxylase). And more particularly still, the LHRR fragment corresponds to nucleotides 57764 to 59291 of the tobacco plastome (Shinozaki et al., 1986—Genbank Z00044). The RHRR fragment corresponds to nucleotides 59299 to 60536 of the tobacco plastome.

To obtain plastid transformation, the transforming DNA must cross the cell wall, the plasma membrane and the double membrane of the organelle before reaching the stroma. In this respect, the most commonly used technique for transforming the plastid genome is that of particle bombardment (Svab and Maliga, 1993, Proc. Natl. Acad. Sci. USA, February 1, 90(3):913-917).

Plastid transfection using high velocity microprojectiles was first performed in the single-celled alga Chlamydomonas reinhardtii (Boynton et al., 1988). Currently, in higher plants, stable transformation of plastids is commonly carried out in tobacco, Nicotiana tabacum (Svab and Maliga, 1990, Proc. Natl. Acad. Sci. USA 87, 8526-8530; Svab and Maliga, 1993, Proc. Natl. Acad. Sci. USA, February 1, 90(3):913-917). Transformation of plastids from rice (Khan M.S, and Maliga, 1999, Nat. Biotechnol. 17, 910-915), from Arabidopsis thaliana (Sikdar et al., 1998, Plant Cell Reports 18:20-24), from potato (Sidorov et al, 1999, Plant J. 19(2):209-216), from Brassica napus (Chaudhuri et al., 1999, WO 00/39313) and from tomato (Ruf et al., 2001, Nat. Biotechnol. 19, 870-875) have been reported. Fertile transplastomic plants have been obtained for tobacco, tomato, potato and soybean (WO 04/053133). Recently, transformation of duckweed plastids has been reported (WO 05/005643).

Selective marker may be used to select for transformed plastids and cells, i.e. those that have incorporated the chimeric gene(s) into their plastome (i.e. transplastomic cells), and it also makes it possible to obtain fertile, homoplasmic transplastomic plants. The term “homoplasmic” means that all the cells contain the same kind of plastome and only that plastome. Transplastomic plants are homoplasmic when all their cells contain only copy of the transformed plastome.

Among the genes that can be used as selective markers, by way of example, special mention can be made of two chimeric genes, namely the aadA gene which codes for an aminoglycoside 3″-adenyltransferase that confers resistance to spectinomycin and streptomycine (Svab et al., 1993, Proc. Natl. Acad. Sci. 90:913-917), and the neo gene which codes for a neomycin phosphotransferase (Carrer et al., 1993, Mol. Gen. Genet. 241:49-56) that confers resistance to kanamycin. Other suitable candidate selective markers include genes that confer resistance to betain aldehyde such as the gene that codes for betain aldehyde dehydrogenase (Daniell et al., 2001, Curr. Genet. 39:109-116), and also genes that confer herbicide tolerance such as the bar gene (White et al., 1990, Nucleic Acid Res. 18(4):1062) which confers resistance to bialaphos, and the EPSPS gene (U.S. Pat. No. 5,188,642) which confers resistance to glyphosate. Alternatively, reporter genes can be used, i.e. genes that codes for readily identified enzymes such as GUS (β-glucuronidase) (Staub J. M. and Maliga P., 1993, EMBO J. 12, 601-606) or the green fluorescent protein (GFP, Sidorov et al., 1999, Plant J. 19(2):209-216), genes coding for pigments, or for enzymes that regulate pigment production. Such genes are described in Patent Applications WO 91/02071, WO 95/06128, WO 96/38567, WO 97/04130 and WO 01/64023.

The gene coding for the selective marker may be the aadA gene which codes for an aminoglycoside 3″-adenyltransferase that confers resistance to spectinomycin and streptomycine (Svab et al., 1993, Proc. Natl. Acad. Sci. 90:913-917).

The present invention therefore also relates to transplastomic plant cells, or transplastomic plants and/or progeny thereof, having integrated into their plastome a nucleic acid molecule comprising linked to one another in a functional fashion in the direction of transcription a promoter sequence which is active in plastids, a nucleic acid sequence encoding a bacterial signal peptide translationnally fused with a heterologous nucleic acid sequence encoding a peptide, and optionally a terminator which is active in the plastids of plant cells.

In an embodiment of the present invention, the nucleic acid sequence encoding a peptide which is fused to the nucleic acid sequence encoding a bacterial signal peptide, is derived from an eukaryotic organism.

The present invention also relates to a transplastomic plant and/or progeny which is a Lemnaceae, a plant from the genus Nicotiana, a potato plant, a tomato plant, a soybean plant, a canola or rape plant, a cotton plant, a rice plant or an algae.

In a particular embodiment, the transplastomic plant of the invention is a tobacco plant.

In a further embodiment, the present invention relates to harvestable parts of plants according to the invention, such as leaves, wherein these harvestable parts contain plant cells according to the invention.

The present invention also relates to a method for the manufacture of transplastomic plants according to the invention wherein

a) a plant cell is transformed with at least one chimeric gene which comprises, linked to one another in a functional fashion in the direction of transcription, a promoter sequence from a plastomic plant gene, a nucleic acid sequence encoding a bacterial signal peptide translationally fused with a heterologous nucleic acid sequence encoding a peptide, and optionally a terminator which is active in the plastids of plant cells.
b) a plant is regenerated from a plant cell obtained in step a) and
c) if necessary, further plants are produced from the plants obtained in step b).

The plant cell obtained in step a) may be regenerated to whole plants according to methods known to the skilled person, as for example using the methods described in “Plant Cell Culture Protocols” 1999, edited by R. D. Hall, Humana Press, ISBN 0-89603-549-2.

The production of further plants according to Step (c) of the method according to the invention can be carried out, for example, by vegetative propagation (for example using cuttings, tubers or by means of callus culture and regeneration of whole plants) or by sexual propagation. Here, sexual propagation preferably takes place under controlled conditions, i.e. selected plants with particular characteristics are crossed and propagated with one another.

The present invention relates further to a method of producing a peptide of interest in the thylakoid lumen of a transplastomic plant cell comprising the following steps:

a) introducing into a plant cell a chimeric gene comprising:
a promoter from a plastomic plant gene, operably linked to
a nucleic acid sequence encoding a bacterial signal peptide translationally fused with a nucleic acid sequence encoding said peptide of interest, and
optionally a terminator which is active in the plastids of plant cells, resulting in a transplastomic plant cell;
   b) placing the transplastomic plant cell under conditions that allow the expression of the chimeric gene, and the subsequent cleavage of the signal peptide.

The present invention relates also to a method of producing a transplastomic plant expressing a peptide of interest in the thylakoid lumen of a transplastomic plant cell comprising the following steps:

a) introducing into a plant cell a construct comprising:
a promoter from a plastomic plant gene, operably linked to
a nucleic acid sequence encoding a bacterial signal peptide translationally fused with a nucleic acid sequence encoding said peptide of interest, and
optionally a terminator which is active in the plastids of plant cells, resulting in a transplastomic plant cell;
   b) placing the transplastomic plant cell in culture under conditions that allow the transcription of the construct, and the subsequent cleavage of the signal peptide, and
   c) selecting the transplastomic plant cell.

In an embodiment of the invention, the method described above comprises the further step of regenerating a plant from the transplastomic plant cell.

In an embodiment of the invention, the nucleic acid sequence encoding a bacterial signal peptide in the methods described above is translationally fused with a heterologous nucleic acid sequence encoding said peptide of interest, which means that this second nucleic acid sequence is not naturally fused with the nucleic acid encoding a bacterial signal peptide.

In an embodiment of the invention, the peptide of interest produced in the thylakoid lumen of a transplastomic plant cell using the methods described above is a non-methionine N-terminus peptide.

The present invention further relates to the above-described method for producing a non-methionine N-terminus peptide in a plant plastid, wherein the non-methionine N-terminus protein is the aprotinin, or a human growth hormone.

The invention further relates to a method for obtaining a disulfide-bond containing peptide or protein of interest in the thylakoid lumen of a transplastomic plant cell, comprising the following steps:

a) introducing into a plant cell a chimeric gene comprising:
a promoter from a plastomic plant gene, operably linked to
a nucleic acid sequence encoding a bacterial signal peptide translationally fused with a nucleic acid sequence encoding said peptide of interest, and
optionally a terminator which is active in the plastids of plant cells, resulting in a transplastomic plant cell;
   b) placing the transplastomic plant cell under conditions that allow the expression of the chimeric gene, and the subsequent cleavage of the signal peptide.

In an embodiment of the invention, the nucleic acid sequence encoding a bacterial signal peptide is translationally fused with a heterologous nucleic acid sequence encoding said peptide of interest.

Human growth hormone, cholera toxin B, human serum albumin, single chain antibodies, human interferon alpha, or alkaline phosphatase, are examples of disulfide-bond containing peptides which can be obtained by the means and methods of the invention.

The present invention further relates to a method for producing a peptide of interest comprising the step of extracting the peptide of interest from a transplastomic plant cell according to the invention, or from a transplastomic plant and/or progeny thereof according to the invention, or from harvestable parts of a transplastomic plant according to the invention.

The present invention further relates to a method for producing a peptide of interest comprising the following steps:

a) producing said peptide of interest in the thylakoid lumen of a transplastomic plant cell using the method defined above, and
b) extracting the peptide of interest from said transplastomic plant cell.

The present invention further relates to a method for producing a peptide of interest comprising the following steps:

a) producing a transplastomic plant expressing said peptide of interest in the thylakoid lumen of a transplastomic plant cell using the method defined above, and
b) extracting the peptide of interest from said transplastomic plant cell.

Preferably, such a method also comprises the step of harvesting the cultivated plants and/or parts of such plants such as leaves before extracting peptide of interest. Most preferably, it further comprises the step of cultivating the plants of the invention before harvesting.

In an embodiment of the present invention, the nucleic acid sequence encoding a bacterial signal peptide used in the methods described above comprises the sequence given under SEQ ID NO 1 or has an identity of at least 50%, at least 60%, at least 70%, 80%, 90% or 95% with the nucleic acid sequence given under SEQ ID NO 1.

In an embodiment of the present invention, the nucleic acid sequence encoding a bacterial signal peptide used in the methods described above comprises a nucleic acid sequence encoding the amino acid sequence given under SEQ ID NO 2 or encoding a peptide the amino acid sequence of which has an identity of at least 70%, at least 80%, at least 90% or 95% with the amino acid sequence given under SEQ ID NO:2;

As revealed by the following examples, which are not in any way limiting, unexpected and remarkable expression and specific activity of the protein of interest has been obtained using the methods and means of the invention.

### Example 1

### Construction of Transformation Vectors Containing the Alkaline Phosphatase Gene with or without a Bacterial Signal Peptide

The phoA sequence including the bacterial signal peptide targeting the periplasm (PhoA-L) was amplified by PCR from E. coli DH5α genomic DNA with the primers EP-PhoA C (5′-ttatttcagccccagagcgg-3′) and EP-PhoATP N (5′-tgaaacaaagcactattgcactggc-3′). The amplified fragment was cloned into pCR4Blunt-Topo (Invitrogen) to obtain the plasmid (pEPA53) and then cloned into the plasmid pAPR04 to giving the tobacco chloroplastic expression vector PCLT 516. Plasmid pAPR04 is a plastid transformation vector which carries a heterologous expression cassette with a spectinomycin resistance (aadA) gene as a selection marker, the psbA promoter (PpsbA) and the rbcL tobacco terminator. This vector allows the targeted integration of the transgene between the rbcL and accD tobacco plastid genes. The left and right homologous recombination regions (LHRR and RHRR) correspond to tobacco plastome fragment 57,769 to 59,296 and 59,304 to 60,540 respectively (Genbank Z00044; Shinozaki et al., 1986, EMBO J. 5:2043-2049).

The phoA gene without the bacterial signal peptide (PhoA-S) was also amplified by PCR from genomic DNA of E. coli DH5α with the primers EP-PhoA C and EP-PhoA N (5′-gggtcatgaggacaccagaaatgcctgt-3′). The amplified fragment was cloned into pCR4Blunt-Topo and subsequently cloned into the vector pCLT516 deleting the peptide signal in front of the phoA gene, to construct the tobacco chloroplastic expression vector pCLT 515.

### Example 2

### Transformation, Selection and Regeneration of Transplastomic Tobacco Plants

Plastid transformation and plant selection were carried out as described by Svab and Maliga (1993, Proc. Natl. Acad. Sci. USA, 90: 913-917). Briefly, sterile tobacco plants were grown in solid MS medium with 30 g/l sucrose. Transformation was carried out by bombarding 4-5 week-old leaves of this plant with gold particles coated with plasmids pCLT515 and pCLT516 using a “particle influx generator” gun (Finer et al., 1992, Plant Cell Rep; 11: 323-328). Following incubation at 24° C. on MS medium for two day, bombarded leaves were cut into small (˜1 cm×1 cm) pieces and then subjected to rounds of selection on MS medium containing 500 μg/ml of spectinomycin. Spectinomycin resistant shoots obtained after about six weeks were transplanted in greenhouse.

The selected events corresponding to vectors pCLT515 and pCLT516 were phenotypically indistinguishable from wild-type tobacco and fully fertile.

### Example 3

### PCR and Southern Blot Analysis

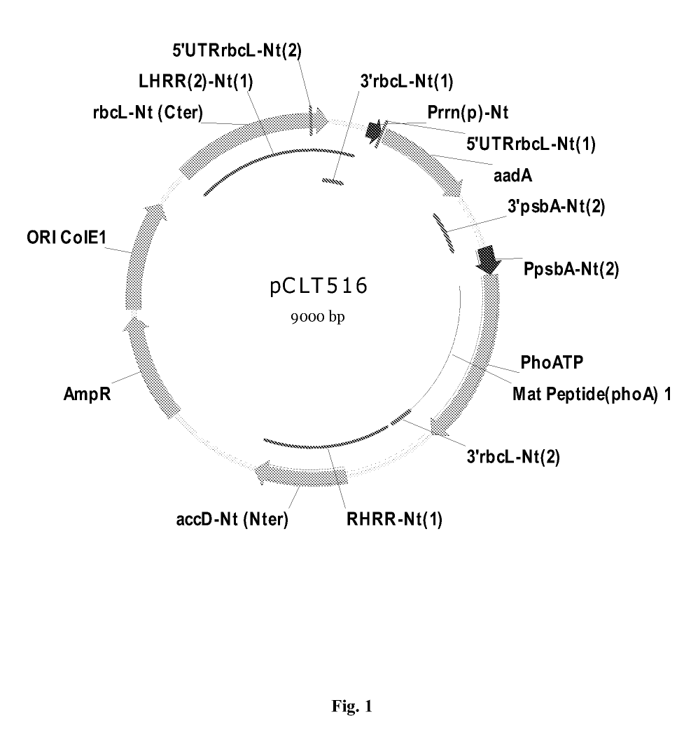

Total plant DNA extraction on T0, T1 and T2 generations was performed using the DNeasy Plant Mini Kit (Qiagen, Courtaboeuf, France). Putative transgenic plants were screened by PCR with different sets of primers: (1) aadA+ (5′-tatggatcccgaagcggtgatc-3′)/(2) aadA-(5′-gatcgctagattatttgccgcta-3′), (5) orbcL52 (5′-atgtcaccacaaacacagactaaagc-3′), and (6) aadA10R (5′-gttgatacttcggcgatcaccgtttc-3′), both sets designed to determine whether the integration of foreign genes had occurred in the chloroplast genome at the targeted site by homologous recombination, and (3) EP-PhoA-1337F (5′-gcatgccgccaatgttgttg-3′)/(4) AccD-V (5′-actgcccattgattatttagccc3′) to demonstrate the presence of the phoA gene in transgenic lines, as described in FIG. 1. PCR reactions were performed in a MJ Research thermocycler using ReadyMix Taq PCR Reaction Mix (Sigma).

For Southern blot analysis 5 μg of total DNA were digested with HindIII, separated on a 0.8% agarose gel and transferred to a nylon membrane. The probes used for the Southern blot analysis, depicted in FIG. 1 were amplified using primer pairs: (i) 5′-gtagagagccgtttatgaatgtcttcg-3′ and 5′-aaggatgtcctaaagttcctccacc-3′ for rbcL, (ii) 5′-gaagcggtgatcgccgaag-3′ and 5′-ttatttgccgactaccttggtgatctcgcc-3′ for aadA, (iii) 5′-ttatttcagccccagagcgg-3′ and 5′-gggtcatgaggacaccagaaatgcctgt-3′ for the phoA and phoATP. The PCR fragments were purified using the PCR purification Kit (Qiagen), and radiolabelled with 32P by random priming with MEGAPRIME Kit (Qiagen). Membranes were washed with 6×SSC, 2×SSC 0.1% SDS and 0, 1×SSC 1% SDS solutions, at 65° C. Autoradiograms were obtained after 2 h exposure, at −80° C., with an intensification screen.

An unique DNA fragment at around 7 kb is detected as expected for events of transformation, confirming the integration of the transgene at the predicted locus between the rbcL and accD plastome genes. No signal is detected with this probe at a higher molecular weight, at around 11500 bp, as expected for wild-type plastome, confirming the homoplasmic stage of the analyzed material. The PhoA probe reveals a major band at around 8 kb, as expected for the transgenic samples, showing that the alkaline phosphatase gene is present in the plastid genome of those lines. Finally, no difference is observed between samples from T0, T1 and T2 generations, showing that the introduced transgenes are stably inherited.

Expression of PhoA and full-length SP-PhoA could easily be detected on 2D-gels by total staining of proteins with silver. The expression of SP-PhoA is dramatically higher than stromal PhoA, and in the same range as the Rubisco large subunit, the most abundant leaf protein.

### Example 4

### Western Blot Analysis and Two-Dimensional Gel Electrophoresis

Transformed and untransformed leaves were frozen in liquid nitrogen and ground to fine powder. Protein extraction buffer (Tris-HCl 50 mM, EDTA mM, DTT 1 mM and protease inhibitor—Complete mini Protease inhibitor cocktail tablets, Roche) was added to the powder, and incubated on ice for 20 min. The mixture was centrifuged at 4° C. at 13,000 g for 5 min. The supernatant, containing total soluble proteins, was removed and protein concentration for each sample was determined by the Bradford Protein Assay Reagent kit (Bio-Rad). An aliquot was taken out, combined with sample loading buffer containing mercaptoethanol, boiled and then run on 12% SDS-PAGE gel. Separated proteins were transferred to a nitrocellulose membrane, using a liquid electroblotting apparatus (Mini-Protean 3 Cell, Bio-Rad). After two hours of saturation at room temperature with TTBS buffer (10×TBS from Bio-Rad, tween 0.1%, Bio-Rad, pH 7,5) containing western blocking reagent (Roche), membranes were washed three times with TTBS buffer and incubated during one night at 4° C. with TTBS buffer containing monoclonal antibody raised in mouse against alkaline phosphatase (mouse anti-Escherichia coli alkaline phosphatase, mAb1012, Chemicon international). After three other washes in TTBS buffer, membranes were incubated two hours at room temperature with TTBS buffer containing secondary antibodies raised in goat against mouse IgGs, (Anti-mouse IgG conjugated to alkaline phosphatase, Sigma). The membranes were revealed after three washes in TTBS buffer and one wash in TBS buffer, with Immun-Star™ AP substrates Pack (Bio-Rad) on Hyperfilm™ ECL (Amersham).

Native PAGE (without SDS) was performed in same condition, with total proteins from leaves extracted in 50 mM Tris-HCL buffer supplement with protease inhibitor (Complete Mini Protease inhibitor cocktail tablets, EDTA free, Roche). The proteins were not boiled and mercaptoethanol was not added to the sample before loading. Proteins were analyzed by 2D gel electrophoresis using techniques described previously (Rajjou et al., 2004, Plant Physiol., 134: 1598-1613; Job et al, 2005, Plant physiol. 38(2): 790-802). Total soluble proteins (150 μg) extracted from leaves of T0 tobacco plastid transformants were separated using gel strips forming an immobilized nonlinear pH gradient from 3 to 10 (Immobiline Dry Strip pH 3-10 NL, 18 cm; Amersham Biosciences). The second dimension was carried out in 10% SDS-polyacrylamide gels. Proteins were revealed with Coomassie blue gel code stain reagent (Pierce) or transferred to a membrane for immunoblotting proceeded as described previously for western blot analysis.

### Example 5

### In Vitro & In Vivo Activity Assay

Alkaline phosphatase activity was detected using NBT/BCIP substrate (NBT/BCIP ready-to-use tablets, Roche). 5-Bromo-4-chloro-3-indolyl Phosphate (BCIP) in combination with Nitro-Blue Tetrazolium Chloride (NBT) yields an intense, insoluble black-purple precipitate in presence of alkaline phosphatase. The NBT/BCIP reaction proceeds at a steady rate, allowing accurate control of the relative sensitivity and control of the development of the reaction.

One tablet of NBT/BCIP in 10 ml of sterile water was used directly on PVDF membranes and the PhoA signal was revealed after 20 minutes of incubation in the dark at room temperature.

Bacterial strains expressing either PhoA or SP-phoA were grown over night at 37° C. on solid LB medium supplemented with antibiotic (500 μg/mL spectinomycin) and with NBT-BCIP (one tablet for 500 ml in LB medium).

For the assay on transformed plants with phoA or SPphoA pieces of tobacco leaves were incubated over night at 37° C. in 10 ml sterile water supplemented with NBT-BCIP (200 μl at 20 mg/ml).

The results show that a very strong alkaline phosphatase activity is detected in transgenic tobacco lines expressing the full-length SP-PhoA protein. The activity of PhoA is clearly detected, but at a lower level than SP-PhoA.

### Example 6

### Estimation of Total Active Protein

pNPP hydrolysis assay of Alkaline Phosphatase—The assay was performed by mixing in each well, of a 96 wells plate, 260 μl, of 100 mM Glycine, 1 mM MgCl2, 1 mM ZnCl2 and different concentrations of total proteins extracted in native condition from bacteria and plants transformed either with pCLT515 or pCLT516. The final volume was adjusted to 280 μl with para-Nitrophenyl Phosphate buffer (pNPP, 1 tablet diluted in 5 ml sterile water, Sigma), that initiates the reaction. The assay was read against blank at 405 nm in a Beckman Coulter AD340 spectrophotometer. The result was obtained by using the equations:

$$\frac{Units of phoA activity}{ml} = \frac{\begin{matrix}( \frac{Δ {A}_{405 n m}}{min Test} - \frac{Δ {A}_{405 n m}}{min Blank} ) \times \\ ( total volume reaction in ml )\end{matrix}}{18.5 \times ( \begin{matrix}volume of enzyme \\ used in ml\end{matrix} )}$$

18.5: Millimolar extinction coefficient of pNPP at 405 nm.

Units of phoA/mg total protein=(Units/ml)/(mg protein/ml enzyme)

Quantification of phoA expressed in bacteria and plants—The extracted proteins were loaded on 12% SDS-PAGE, the revelation of the phoA signal was performed directly on PVDF membranes with Immun-Star™ AP substrates by chemiluminescence. Protein concentration for each sample (mg of phoA/mg total protein) was determined on the revealed membranes using the Quantity one software from Bio-Rad.

Specific activity—To calculate the specific activity of phoA we used both pNPP activity and quantification of phoA. With the ratio of the two results we obtain the quantity of active phoA for a determined quantity of phoA expressed in transformants. These results show that SP-PhoA is not only more highly expressed in chloroplasts than PhoA, but that it is also expressed in a more active form which has a significantly higher specific activity, similar to that found for SP-PhoA in bacterial periplasm.

### Example 7

### Protein Localization

Chloroplast fractionation—Tobacco leaves were blended twice for 2-3 s in 330 mM sorbitol 50 mM Hepes-KOH (pH 7.8), 10 mM KCl, 1 mM EDTA, 0.15% (w/v) bovine serum albumin, 4 mM sodium ascorbate, and 7 mM cysteine. The resulting mixture was filtered through three layers of nylon mesh (20 μm), and the filtrate was centrifuged 20 min at 3000 rpm at 4° C. The pellets were resuspended in 25 ml of 330 mM sorbitol, 50 mM Hepes-KOH (pH 7.8), 10 mM KCl, centrifuged for 20 min at 3000 rpm at 4° C., and resuspended in 25 ml of the same buffer. The suspension obtained was layered over a two-step gradient Percoll (8 ml of 40%, 4 ml of 80%). The gradients were centrifuged at 3700 rpm for 10 minutes at 4° C. The lower green band containing intact plastids was collected and diluted in 10 mM sodium pyrophosphate buffer (pH 7.8). Thylakoids were then collected by centrifugation on a sucrose gradient (15 ml saccharose 0.6M) at 15000 rpm for 10 min at 4° C. The supernatant containing the stromal extract was separated from the pellet containing intact chloroplasts. The latter was washed twice with each of the following buffers: (i) 10 mM sodium pyrophosphate (pH 7.8) to remove residual stromal proteins; (ii) 2 mM Tricine (pH 7.8), 300 mM sucrose to remove unidentified extrinsic thylakoid membrane proteins; (iii) 30 mM sodium phosphate (pH 7.8), 50 mM NaCl, 5 mM MgCl2, and 100 mM sucrose (fragmentation buffer). Intact thylakoids were further fractionated by sonication on ice, three times for 10 s each. The sonicated thylakoids were then centrifuged for 5 minutes in an eppendorf centrifuge at 4° C. to remove intact thylakoids, and thylakoids membranes were separated from the lumen extract by ultracentrifugation of the supernatant.

Alkaline phosphatase localization—All fractions obtained were subjected to western blot analysis using antibodies against both a plastid soluble lumenal protein TL 29 (antibody used at a 1/10000 dilution), a plastid soluble stromal protein KARI (used at 1/10000 dilution) and alkaline phosphatase (used as described before).

Our results show that PhoA and SP-PhoA do not have the same distribution within chloroplasts. PhoA is essentially detected in the stromal compartment, as expected. A substantial proportion of SP-PhoA is detected in the thylakoid lumen fraction, showing that the bacterial signal peptide is able to translocate alkaline phosphatase across the thylakoid membrane. The higher specific activity of SP-PhoA is therefore attributed to the localization of this enzyme in the lumen of thylakoids, which is more appropriate than the stroma for high-level accumulation of active and correctly folded enzyme.

### Example 8

### Targeting of a Heterologous GUS Protein to the Thylakoid Lumen

A gene fusion between the alkaline phosphatase signal peptide and the GUS reporter enzyme (beta-glucuronidase from E. coli) was made. A synthetic gene (SEQ ID NO 3) was ordered which contains from the 5′ to 3′ end the sequence of alkaline phosphatase signal peptide starting at the second codon (the codon encoding the N-terminal methionine of the signal peptide will be restored by cloning inside the final transformation vector) fused to the 5′ sequence of the GUS gene from the second codon (gtc for Valine) to the Mscl restriction site. This sequence was inserted in the cloning vector pCR-TOPO-Blunt (Invitrogen), giving vector PCLT-PhoA-GUS Nter. The cloned 337 bp fragment obtained after restriction with DraI and Mscl was then cloned in the tobacco plastid transformation vector pCLT194 digested with Ncol (filled in) and Mscl. The tobacco transformation vector pCLT-PhoA-GUS contains the translational fusion between the signal peptide of alkaline phosphatase (PhoA) and the GUS coding region. In pCLT194, This open reading frame is placed under the control of the tobacco plastid psbA promoter and targets recombinant GUS in the thylakoid lumen whereas GUS expressed from pCLT194 in tobacco plastid transformants accumulates GUS in the stroma.

1. A chimeric gene comprising, linked to one another in a functional fashion in the direction of transcription:
a) a promoter from a plastomic plant gene,
b) a nucleic acid sequence encoding a bacterial signal peptide translationally fused with,
c) a heterologous nucleic acid sequence encoding a peptide of interest,
d) optionally a terminator which is active in the plastids of plant cells.

2. A vector designated for the transformation of plant plastids, characterized in that it contains at least two sequences that are homologous to sequences in the plastome of the plant to be transformed, said homologous sequences flanking at least one chimeric gene according to claim 1.

3. A transplastomic plant cell, characterized in that it contains at least one chimeric gene according to claim 1.

4. A transplastomic plant or progeny thereof comprising a transplastomic plant cell according to claim 3.

5. A transplastomic plant or progeny thereof according to claim 4, which is an alga, a Lemnaceae, a plant from the genus Nicotiana, a potato, tomato, rape, canola rice, cotton or soybean plant.

6. Harvestable parts of a plant comprising transplastomic plant cells according to claim 3.

7. A method for the manufacture of a transplastomic plant or progeny thereof according to claim 4 comprising:
a) transforming a plant cell with at least one chimeric gene which comprises, linked to one another in a functional fashion in the direction of transcription, a promoter sequence which is active in plastids, a nucleic acid sequence encoding a bacterial signal peptide translationally fused with a heterologous nucleic acid sequence encoding a peptide, and optionally a terminator which is active in the plastids of plant cells;
b) regenerating a plant from a plant cell obtained in step a) and
c) optionally, producing further plants from the plants obtained in step b).

8. A method of producing a peptide of interest in the thylakoid lumen of a transplastomic plant cell comprising the following steps:
a) introducing into a plant cell a chimeric gene comprising:
a promoter that is functional in a plant plastid, operably linked to
a nucleic acid sequence encoding a bacterial signal peptide translationally fused with a nucleic acid sequence encoding said peptide of interest, and
optionally a terminator which is active in the plastids of plant cells, resulting in a transplastomic plant cell; and
b) placing the transplastomic plant cell under conditions that allow the expression of the chimeric gene, and the subsequent cleavage of the signal peptide.

9. A method of producing a transplastomic plant expressing a peptide of interest in the thylakoid lumen of a transplastomic plant cell comprising the following steps:
a) introducing into a plant cell a construct comprising:
a promoter that is functional in a plant plastid, operably linked to
a nucleic acid sequence encoding a bacterial signal peptide translationally fused with a nucleic acid sequence encoding said peptide of interest, and
optionally a terminator which is active in the plastids of plant cells, resulting in a transplastomic plant cell;
b) placing the transplastomic plant cell under conditions that allow the expression of the chimeric gene, and the subsequent cleavage of the signal peptide, and
c) selecting the transplastomic plant cell.

10. The method of claim 9, comprising the further step of regenerating a plant from the transplastomic plant cell.

11. The method of claim 8 wherein said peptide of interest is a non-methionine N-terminus peptide.

12. (canceled)

13. A method for producing a peptide of interest comprising the following steps:
a) producing said peptide of interest in the thylakoid lumen of a transplastomic plant cell using the method defined in claim 8, and
b) extracting the peptide of interest from said transplastomic plant cell.

14. A method for producing a peptide of interest comprising the following steps:
a) producing a transplastomic plant expressing said peptide of interest in the thylakoid lumen of a transplastomic plant cell using the method defined in claim 9, and
b) extracting the peptide of interest from said transplastomic plant cell.

15. (canceled)

16. (canceled)

17. (canceled)

18. The method of claim 8 wherein said protein of interest is a peptide containing a disulfide bond.

19. The method of claim 9 wherein said peptide of interest is a non-methionine N-terminus peptide.

20. The method of claim 10 wherein said peptide of interest is a non-methionine N-terminus peptide.


SEQUENCE LISTING (18 sequences)

SEQ ID NO: 1 - DNA, 63 bp, Escherichia coli
  sig_peptide (1)..(12)
gtgaaacaaagcactattgcactggcactcttaccgttactgtttacccctgtgacaaaagcc

SEQ ID NO: 2 - protein, 21 aa, Escherichia coli
  SIGNAL (1)..(21)
MKQSTIALALLPLLFTPVTKA

SEQ ID NO: 3 - DNA, 685 bp, Artificial
  synthetic sequence comprising a truncated sequence encoding the PhoA signal peptide from E. coli fused to the truncated beta-glucuronidase gene from E. coli
tttaaacaaagcactattgcactggcactcttaccgttactgtttacccctgtgacaaaagccgtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggcca

SEQ ID NO: 4 - DNA, 20 bp, Artificial
  synthetic primer
ttatttcagccccagagcgg

SEQ ID NO: 5 - DNA, 25 bp, Artificial
  synthetic primer
tgaaacaaagcactattgcactggc

SEQ ID NO: 6 - DNA, 28 bp, Artificial
  synthetic primer
gggtcatgaggacaccagaaatgcctgt

SEQ ID NO: 7 - DNA, 22 bp, Artificial
  synthetic primer
tatggatcccgaagcggtgatc

SEQ ID NO: 8 - DNA, 23 bp, Artificial
  synthetic primer
gatcgctagattatttgccgcta

SEQ ID NO: 9 - DNA, 26 bp, Artificial
  synthetic primer
atgtcaccacaaacacagactaaagc

SEQ ID NO: 10 - DNA, 26 bp, Artificial
  synthetic primer
gttgatacttcggcgatcaccgtttc

SEQ ID NO: 11 - DNA, 20 bp, Artificial
  synthetic primer
gcatgccgccaatgttgttg

SEQ ID NO: 12 - DNA, 23 bp, Artificial
  synthetic primer
actgcccattgattatttagccc

SEQ ID NO: 13 - DNA, 27 bp, Artificial
  synthetic primer
gtagagagccgtttatgaatgtcttcg

SEQ ID NO: 14 - DNA, 25 bp, Artificial
  synthetic primer
aaggatgtcctaaagttcctccacc

SEQ ID NO: 15 - DNA, 19 bp, Artificial
  synthetic primer
gaagcggtgatcgccgaag

SEQ ID NO: 16 - DNA, 30 bp, Artificial
  synthetic primer
ttatttgccgactaccttggtgatctcgcc

SEQ ID NO: 17 - DNA, 20 bp, Artificial
  synthetic primer
ttatttcagccccagagcgg

SEQ ID NO: 18 - DNA, 28 bp, Artificial
  synthetic primer
gggtcatgaggacaccagaaatgcctgt